(19) United States
(12) Patent Application Publication
(10) Pub. No.: US 2019/0271707 A1
(43) Pub. Date: Sep. 5, 2019

(54) Method and Apparatus Employing Magnetic Beads for Ligand Binding Assays of Biological Samples

(71) Applicant: Virgin Instruments Corporation, Marlborough, MA (US)

(72) Inventor: Marvin L. Vestal, Framingham, MA (US)

(73) Assignee: Virgin Instruments Corporation, Marlborough, MA (US)

(21) Appl. No.: 15/910,064

(22) Filed: Mar. 2, 2018

(51) Int. Cl. G01N 33/68; G01N 33/53

(52) U.S. Cl. CPC G01N 33/6851; H01J 49/0431; G01N 33/5308

(57) ABSTRACT

An apparatus for ligand binding of biological samples includes a bead well configured to confine a plurality of magnetic beads. A sample well comprises a filter bottom configured to contain samples of interest. A first magnetic bead picker captures magnetic beads from the bead well and releases the captured magnetic beads into the sample well. An incubator incubates the magnetic beads in the sample well binding the bait molecules to sample molecules contained in the sample of interest. A washer washes the incubated magnetic beads removing weakly bound sample molecules while retaining magnetic beads comprising strongly bound sample molecules. A second magnetic bead picker captures the magnetic beads comprising strongly bound sample molecules from the sample well and releases the captured magnetic beads comprising strongly bound samples onto a sample plate. A matrix material applicator deposits MALDI matrix material onto a surface of the sample plate. A MALDI-TOF mass spectrometer receives the sample plate with deposited MALDI matrix material and performs time-of-flight mass spectrometry on the strongly bound sample molecules, thereby generating mass spectra of the sample. A computer executes an algorithm using the mass spectra generated by the MALDI-TOF mass spectrometer to produce a ligand binding assay.

### CROSS REFERENCE TO RELATED APPLICATION

The present application is related to U.S. patent application Ser. No. 15/861,265, entitled “Ligand Binding Assays Using MALDI-TOF Mass Spectrometry” filed Jan. 3, 2018, which claims priority to U.S. Provisional Patent Application Ser. No. 62/442,512, entitled “Ligand Binding Assays Using MALDI-TOF Mass Spectrometry” filed on Jan. 5, 2017. The present application is also related to U.S. patent application Ser. No. 15/079,900, entitled “MALDI-TOF MS Method And Apparatus For Assaying An Analyte In A Bodily Fluid From A Subject”, which claims priority to U.S. Provisional Patent Application Ser. No. 62/139,885, entitled “MALDI-TOF MS Method And Apparatus For Assaying An Analyte In A Bodily Fluid From A Subject” filed on Mar. 30, 2015. The entire contents of U.S. patent application Ser. Nos. 15/861,265 and 15/079,900 and U.S. Provisional Patent Application Ser. Nos. 62/442,512 and 62/139,885 are herein incorporated by reference.

The section headings used herein are for organizational purposes only and should not to be construed as limiting the subject matter described in the present application in any way.

### INTRODUCTION

Recent discoveries of disease biomarkers and the establishment of mass spectrometers suitable for clinical applications have led to a recognition that automated prediction, diagnosis, and management of diseases is a realistic short term goal. Early diagnosis has obvious benefits in that it allows physicians to begin treatments sooner. Also, properly identifying disease and disease sub-classification allow physicians to tailor treatments to specific patients, thereby greatly improving outcomes.

Major research efforts are focusing on characterizing the millions of interactions of the human proteome with other molecules. These include proteins, nucleic acids, lipids, and metabolites. Immunoassays are important tools that are used to perform this work. Factors such as: (1) the rising incidences of chronic and infectious diseases; (2) the rapidly expanding biotechnology and pharmaceutical industries; (3) the extensive use of immunoassays in oncology because of its cost-effectiveness and rapid action; and (4) the growing geriatric population are expected to propel the growth of the immunoassay market in the coming years. See, for example, Genetic and Engineering &Biotechnology News, Sep. 15, 2016, p. 12. It is, however, highly desirable to have an alternative to the widely used Enzyme-Linked Immunosorbent Assays (ELISA) that is significantly faster, more sensitive, and less expensive than known methods.

Ligand binding assays have been used to measure the interactions that occur between two molecules, such as protein-bindings, as well as the degree of affinity for which the reactants bind together. More specifically, ligand binding assays are used to test for the presence of target molecules in a sample that is known to bind to the receptor. Various detection methods have been used to determine the presence and extent of the ligand-receptor complexes formed. For example, known methods include electrochemical detection through various fluorescence detection methods.

### BRIEF DESCRIPTION OF THE DRAWINGS

The present teaching, in accordance with preferred and exemplary embodiments, together with further advantages thereof, is more particularly described in the following detailed description, taken in conjunction with the accompanying drawings. The skilled person in the art will understand that the drawings, described below, are for illustration purposes only. The drawings are not necessarily to scale, emphasis instead generally being placed upon illustrating principles of the teaching. The drawings are not intended to limit the scope of the Applicant's teaching in any way.

FIG. 1 illustrates a workflow diagram for an embodiment of an apparatus and method for ligand binding assays according to the present teaching.

FIG. 2A illustrates a front cut-away view of an embodiment of a magnetic bead picker of the present teaching.

FIG. 2B illustrates a side-view of the embodiment of the magnetic bead picker of FIG. 2A.

FIG. 3A illustrates an expanded view of a lower section of an embodiment of a well that is part of a plate preparation apparatus that uses a magnetic bead picker of the present teaching.

FIG. 3B illustrates a table that provides approximate bead capture volume dimensions for various embodiments of the magnetic bead picker according to the present teaching.

FIG. 4A illustrates an embodiment of part of a plate preparation apparatus of the present teaching at different points during processing.

FIG. 4B illustrates the steps of an embodiment of a method that is associated with the apparatus of FIG. 4A.

FIG. 5 illustrates a schematic diagram of an embodiment of a bead washer/incubator according to the present teaching.

FIG. 6A illustrates an embodiment of part of a plate preparation apparatus of the present teaching at different points during processing from bead pick-up through preparation for washing and incubation.

FIG. 6B illustrates the steps of an embodiment of a method that is associated with the apparatus of FIG. 6A.

FIG. 7A illustrates an embodiment of portion of a plate preparation apparatus of the present teaching at different points during processing for incubating and washing the mixture of beads plus sample.

FIG. 7B illustrates the steps of an embodiment a method associated with the apparatus of FIG. 7A.

FIG. 8A illustrates an embodiment of portion of a plate preparation apparatus of the present teaching at different points during processing for picking up beads through applying a MALDI matrix.

FIG. 8B illustrates the steps of an embodiment of a method associated with the apparatus of FIG. 8A.

FIG. 9A illustrates another embodiment of a portion of a plate preparation apparatus of the present teaching at different points during processing for picking up beads through applying a MALDI matrix.

FIG. 9B illustrates the steps of an embodiment of a method associated with the apparatus of FIG. 9A.

FIG. 10 illustrates an embodiment of a high-volume automated MALDI plate preparation apparatus of the present teaching.

FIG. 11 illustrates an embodiment of a MALDI sample plate of the present teaching.

FIG. 12 illustrates an embodiment of a gasket of the present teaching.

FIG. 13A illustrates a diagram of a well layout for a microwell plate of the present teaching that accommodates ˜0.04-mm-diameter beads.

FIG. 13B shows a detailed view of one particular hexagon in the hexagonal array layout shown in FIG. 13A.

FIG. 14 illustrates a table that presents the probability of missing one of n distinguishable beads in a collection of m for various values of m/n and n as applies to an embodiment of the system of the present teaching.

FIG. 15A illustrates a front-view of an embodiment of a MALDI sample plate that can accommodate a large number of beads of the present teaching.

FIG. 15B illustrates a side-view of a section of the MALDI sample plate of FIG. 15A.

FIG. 16A illustrates a top-view of an embodiment of a cassette comprising four MALDI sample plates that can accommodate a large number of beads according to the present teaching.

FIG. 16B illustrates an end-view of the cassette comprising four MALDI sample plates of FIG. 16A.

### DESCRIPTION OF VARIOUS EMBODIMENTS

The present teaching will now be described in more detail with reference to exemplary embodiments thereof as shown in the accompanying drawings. While the present teachings are described in conjunction with various embodiments and examples, it is not intended that the present teachings be limited to such embodiments. On the contrary, the present teachings encompass various alternatives, modifications and equivalents, as will be appreciated by those of skill in the art. Those of ordinary skill in the art having access to the teaching herein will recognize additional implementations, modifications, and embodiments, as well as other fields of use, which are within the scope of the present disclosure as described herein.

Reference in the specification to “one embodiment” or “an embodiment” means that a particular feature, structure, or characteristic described in connection with the embodiment is included in at least one embodiment of the teaching. The appearances of the phrase “in one embodiment” in various places in the specification are not necessarily all referring to the same embodiment.

It should be understood that the individual steps of the methods of the present teachings can be performed in any order and/or simultaneously as long as the teaching remains operable. Furthermore, it should be understood that the apparatus and methods of the present teachings can include any number or all of the described embodiments as long as the teaching remains operable.

Mass spectrometry (MS) has significantly advanced human protein biomarkers research. However, for all its innovative aspects in protein analysis, MS has yet to make a significant inroad into clinical laboratories and find use its use in protein biomarker diagnostic applications. The steps preceding the MS analysis, from biological sample to protein introduction into the mass spectrometer, are the bottleneck for today's MS protein tests. The present teaching relates to simpler, faster, and cheaper sample preparation workflows that will facilitate clinical MS protein tests adoption. One aspect of the present teaching is that the apparatus and methods of the present teaching can transform cancer research by enabling fast and cost-effective screening of protein biomarkers and clinically relevant proteoforms that may have significant implications in cancer diagnostics and therapy monitoring.

The solution described in connection with the present teaching includes bead-based immunoaffinity capture, with the straightforward MS detection offered by matrix assisted laser desorption ionization (MALDI) based analysis. In particular, matrix assisted laser desorption ionization time-of-flight mass spectrometry (MALDI-TOF MS) can be used. Some steps in this workflow are the transfer of beads to a MALDI sample plate and the release of the captured proteins and, in an efficient, non-dilutive, and sample-loss minimizing fashion that results in millimeter-size sample spots on the MALDI target.

Another aspect of the present teaching is that the apparatus and methods of the present teaching can be used to quickly determine the concentration of biomarkers with concentrations below the current detection threshold of many state-of-the-art MALDI instruments. When predetermined components are present at a low level in a sample of blood or other bodily fluid, a larger volume of analyte may be required to obtain a sufficient number of biomarker molecules for detection and precise quantification. The concentration of the target marker(s) can be increased by conventional methods known in the art. Such methods include one or more of drying, evaporation, centrifugation, sedimentation, precipitation, differential mobility or retention, ion exchange and amplification. A particularly powerful method of enrichment employs an appropriate antibody to capture a specific component of interest. The antibody may be covalently bound to the bead. An example of a targeted analyte that requires concentration is the biomarker and diagnostic substance known as troponin, which is commonly used for the diagnosis of various heart disorders. Functional or healthy troponin occurs as a complex of three subunits that are distinguished by name as troponin C, troponin I, and troponin T. Physiologically, the troponin complex is involved in the contraction of cardiac and skeletal muscle diseases. More specifically, measurement and quantification of troponin subtypes T and I in blood are used as indicators of damage to heart muscle. These measurements are diagnostic, and are used to differentiate between unstable angina and myocardial infarction (heart attack) in people with chest pain or acute coronary syndrome. In addition, non-thrombotic cardiac conditions (myocardial contusion, infiltrative myocardial diseases) and non-thrombotic diagnoses (sepsis, pulmonary embolism, stroke, renal failure) are also associated with elevated levels of troponin.

The current prognostic threshold for troponin T in blood is 0.01 ug/L or ˜1 pmol/L (1×10¹² M). This concentration is below the current detection threshold of many state-of-the-art MALDI instruments, particularly if the molecule remains as a minor constituent in blood. However, affinity-capture chemistry employing target bound antibodies for the specific extraction and concentration of troponin can enable detection and quantification. Inclusion of a synthetic troponin analogue, or a labeled form of troponin with heavy isotopes, can be used to make quantitative measurements. One skilled in the art can estimate relative protein concentrations using protein affinity purification with antibodies, the employment of synthetic isotopically labeled controls, and the incorporation of a calibration curve.

The present teaching describes a system and method for ligand binding assay of biological samples. The system uses a plurality of magnetic beads with bait molecules attached to each bead. The beads are positioned within a sample well that is part of a sample plate. A magnetic bead picker picks magnetic beads from a bead well in a predetermined volume. The magnetic bead picker then releases the magnetic beads into a sample well that contains samples of interest. The samples may be obtained from bodily fluids that include, but are not restricted to, blood and blood products (serum, plasma, platelets), ascites fluid, breast milk, cerebrospinal fluid, lymph fluid, saliva, urine, gastric and digestive fluid, tears, stool, semen (and semen-derived fluids such as aspermic semen), prostatic fluid, vaginal fluid, amniotic fluid, and interstitial fluids derived from tissue.

In some embodiments, the samples of interest and the magnetic beads are already positioned together within a well of a sample plate, and, as such, a magnetic bead picker is not used to move the predetermined volume of magnetic beads to a well that includes a sample. However, the magnetic bead picker may pick up a predetermined volume of beads plus sample, and transfer these beads plus sample to another test plate for analysis, as required by the sample preparation process.

In other embodiments, the magnetic bead picker is used to pick a predetermined volume of beads plus sample and place the beads plus sample into a well positioned on a sample plate that is suitable for washing and incubation. The washing and incubation binds the bait molecule to the sample molecule, and washes away any weakly bound molecules. For example, in some methods, washing includes washing with a Tris pH 7.3 buffer solution. The washed and incubated beads are then loaded using a magnetic bead picker onto a MALDI sample plate containing wells. In some embodiments the wells are sized to allow only one bead per well. In some embodiments, multiple beads are placed in a single well. In other embodiments with multiple beads per well, a multiplexing analysis technique is employed to be able to provide ligand binding assay on multiple analytes of interest simultaneously or nearly simultaneously.

A MALDI matrix solution can be added to the loaded beads plus sample. Then, a matrix assisted laser desorption ionization time-of-flight (MALDI-TOF) mass spectrometer receives the loaded sample plate and performs time-of-flight mass spectrometry to generate mass spectra. A computer executes an algorithm using the mass spectra generated by the MALDI-TOF mass spectrometer to produce a ligand binding assay.

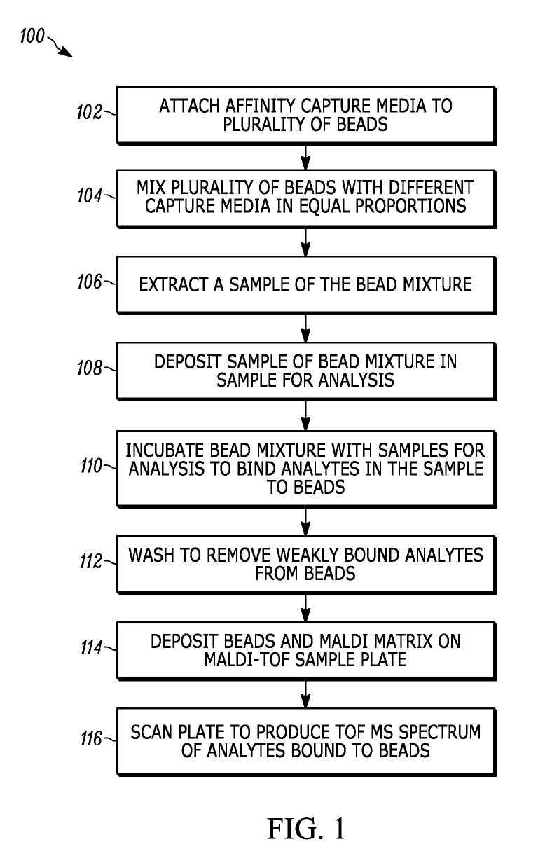

FIG. 1 illustrates a workflow diagram 100 for an embodiment of an apparatus and method for producing ligand binding assays according to the present teaching. In a first step 102 of the method 100, one or more affinity capture media are attached to a plurality of beads. The affinity capture media may include any of the known media used to capture biological and/or chemical molecules, including proteins. Then, in a second step 104 of the method 100, the plurality of beads with one or more capture media are mixed in specified proportion. In some embodiments, equal proportions of beads of each different capture media are mixed. In some embodiments, the capture media is a bait molecule. In a third step 106 of the method 100, a sample of the bead mixture generated in step two 104 is extracted. The sample extracted in step three 106 is then deposited in a target test sample in a fourth step 108 of method 100. The test sample is incubated in the fifth step 110 to bind analytes to the beads. The test sample incubated in step five 110 is then washed in a sixth step 112 of the method 100 to remove weakly bound analytes. In a seventh step 114 of the method 100, the washed beads are deposited onto a sample plate and a MALDI matrix is applied to produce a MALDI-TOF sample plate. In an eighth step 116 of method 100, a MALDI-TOF scan is performed to produce a TOF mass spectrum of the analytes that are bound to the beads.

In some embodiments, the method includes steps for assaying an analyte in a bodily fluid from a subject. See, for example, U.S. patent application Ser. No. 15/079,900, entitled “MALDI-TOF MS Method And Apparatus for Assaying an Analyte in a Bodily Fluid From a Subject”, which is assigned to the present assignee, and which has been incorporated herein by reference. For example, the method may include saving only mass spectra that exceed a predetermined intensity level, and/or determining the mass-to-charge ratios from the saved spectra, and/or analyzing the mass-to-charge ratios to interpret a resulting mass spectrum. Ionizing light pulses from the MALDI-TOF spectrometry may be scanned over a predetermined area of the sample plate, and/or saved spectra may be averaged over a sample spot. Mass spectrometry may be performed by irradiating a spot on the sample plate with a plurality of light pulses and/or the number of the plurality of light pulses may be chosen to reduce noise, and/or achieve a desired level of reproducibility.

One feature of the present teaching is the use of magnetic beads to improve the sample preparation. In some embodiments, the magnetic beads are Sepharose beads with a magnetic core. Bait molecules can be attached to the magnetic beads by variety of techniques. Also, a mass tag molecule may be attached to assist in identifying these beads by mass spectrometry. For example, the mass tag molecules and bait molecules may be biotinylated and bound to the beads by the Streptavidin-biotin interaction. The bait molecules may comprise biotinylated aptamers or biotinylated peptides. Biotinylation is a process of covalently attaching biotin to a protein, nucleic acid, or other molecule. Suitable beads are commercially available from a variety of sources including GE Healthcare and Cube Biotech. The beads are available in a range of sizes from about 1 μm up to 1 mm. In some embodiments, the beads used are nominally 40 μm in diameter. In other embodiments, the beads used are nominally 400 μm in diameter and typically range in size from 350 μm to 450 μm. Known methods of processing magnetic beads use magnets to keep beads in a volume while liquid is introduced and removed to incubate or wash the beads. However, it should be understood that the present teaching expands the use of magnetic beads to provide a more efficient, controlled sample preparation apparatus and method that is suitable for high-volume manufacture.

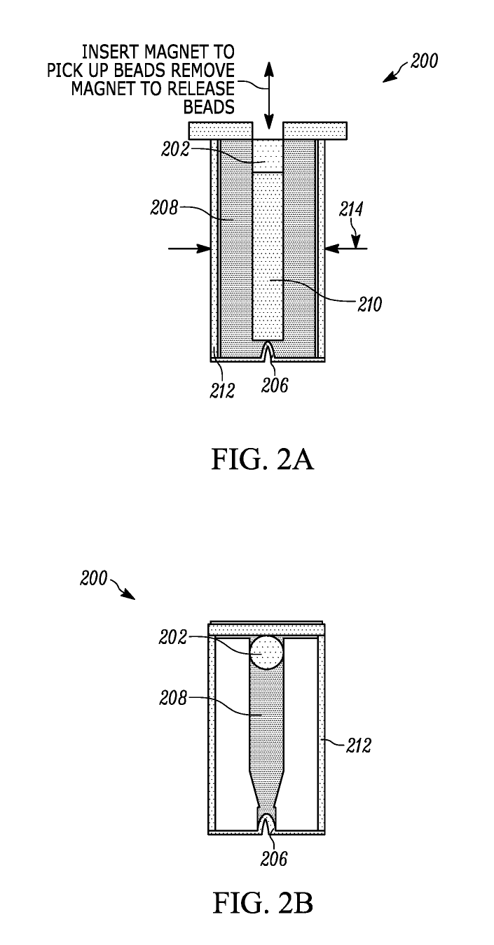

One feature of using magnetic beads is that a magnetic bead picker may be used to remove beads from one volume and introduce beads into another volume. FIG. 2A illustrates a front cut-away view of an embodiment of a magnetic bead picker 200 of the present teaching. FIG. 2B illustrates a side-view of the embodiment of the magnetic bead picker 200 of FIG. 2A. Referring to both FIGS. 2A and 2B, the bead picker 200 uses a magnet 202 that is inserted in the direction 204 shown to pick up beads and removed along the direction 204 shown to release beads. Alternatively, the magnet 202 may be an electromagnet that is activated by a current to pick up beads and deactivated by turning off a current to release beads (not shown). Regardless of whether the magnet 202 is a permanent magnet or an electromagnet, the magnetic bead picker 200 comprises a bead capture volume 206 and a pair of arms formed from soft magnetic material 208 to concentrate the magnetic field in the bead capture volume 206. The beads are thus captured by the concentrated magnetic field from a liquid suspension (not shown) into the bead capture volume 206. The arms of soft magnetic material 208 are confined using an internal plastic spacer 210. A plastic shield 212 surrounds the outside of the magnetic bead picker 200. In some embodiments, a dimension 214, x, of the plastic shield is approximately six millimeters or less. The size of the bead capture volume 206 may be chosen to accommodate a predetermined number of beads.

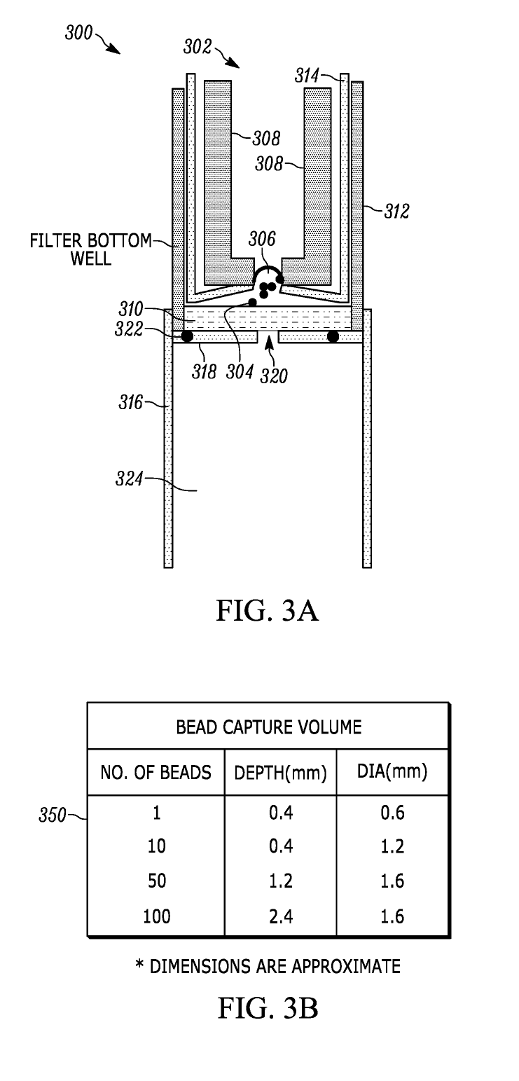

FIG. 3A illustrates an expanded-view of a lower section of an embodiment of a well that is part of a plate preparation apparatus 300 that uses a magnetic bead picker 302 of the present teaching. FIG. 3A illustrates beads 304 moving to occupy the bead capture volume 306. The bead capture volume 306 has dimensions chosen to capture a specified number of beads.

FIG. 3B illustrates a table 350 that provides approximate bead capture volume dimensions for various embodiments of the magnetic bead picker of the present teaching. FIG. 3A illustrates beads 304 occupying the bead volume 306. The beads 304 are attracted by the magnetic field generated by magnet (not shown) and directed by the arms formed of a soft magnetic material 308. The beads 304 are retained on filter 310 at the bottom of a well 312. The plastic shield 314 surrounds the arms of soft magnetic material 308. In some embodiments, the magnetic material may be iron or an iron-containing compound. A lower manifold 316 has a diaphragm 318 with an aperture 320 positioned under the bead capture volume 306. An o-ring 322 is positioned under the well 312 and on top of the diaphragm 318. In some embodiments, the aperture 320 is on the order of 1 mm in diameter or smaller and located at the top of the lower manifold 316 so that the beads are concentrated near the center as liquid flows from the sample well into a lower chamber 324 of the lower manifold 316.

In some embodiments of the methods of the present teaching, the actual number of beads in a bead volume is determined empirically, rather than prescribed by specific dimensions of the volume. In these embodiments, the actual bead numbers captured will be determined empirically in the volumes and adjusted as required.

One feature of the present teaching is that one or more magnetic bead pickers can be used in multiple steps of a process of preparing a MALDI sample plate for MALDI-TOF analysis. In one embodiment, samples are provided in a plate that includes one or more wells with filters at the bottom of each well that retains the beads and allows liquid to flow through. Beads with bait molecules attached may be supplied in separate plates containing wells, where each well may contain a large number of beads that may represent a number of different bait molecules. In some embodiments, the sample plate contains 96 separate wells. Other embodiments use sample plates with different numbers and sizes of wells.

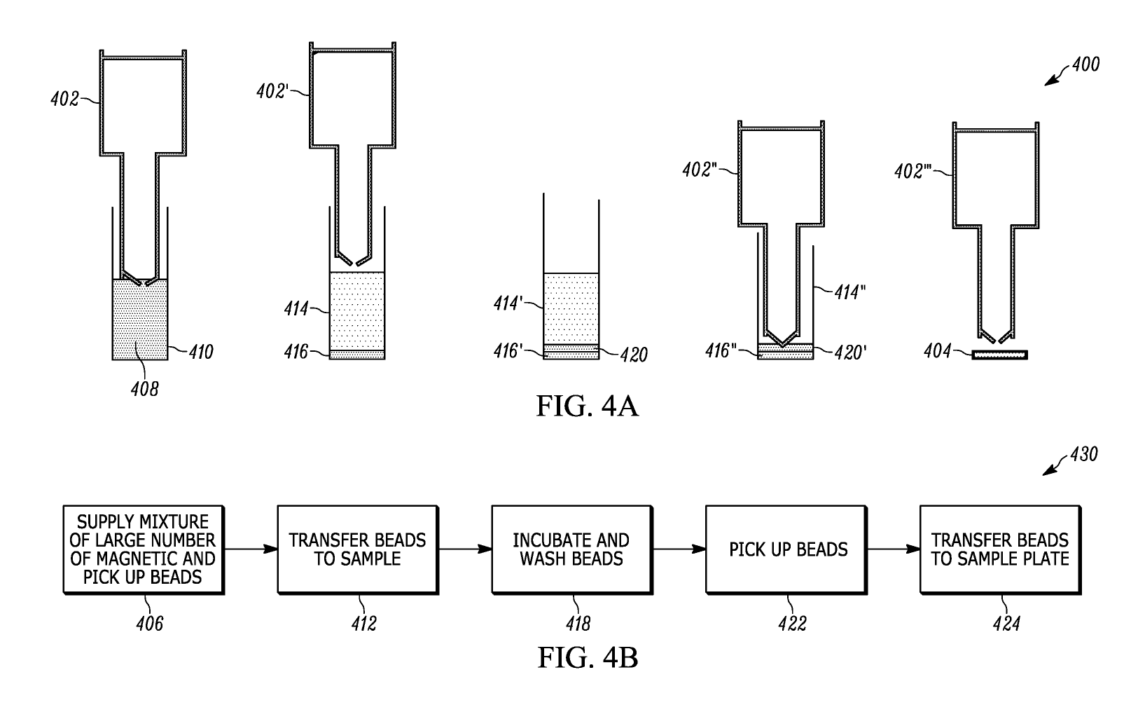

FIG. 4A illustrates an embodiment of part of a plate preparation apparatus 400 of the present teaching at different points during processing. The plate preparation apparatus 400 uses a magnetic bead picker 402, 402′, 402″, 402′″ to prepare a MALDI sample plate 404. FIG. 4B illustrates the steps of an embodiment of a method 430 that is associated with the apparatus of FIG. 4A. Referring to both FIGS. 4A-B, in a first step 406, a mixture 408 containing a large number of beads is supplied in a well 410. In some embodiments, the mixture contains a large number of beads with different antibodies attached. In some embodiments, the number of 0.4 mm beads in 1 mL of bead slurry is approximately 10,000 beads. In these embodiments, an equal number of 50 different antibodies, 20 microliters each, making up the 1 mL of bead slurry. This configuration corresponds to approximately 200 beads per antibody.

As shown in FIGS. 4A-B, in a first step 406, the magnetic bead picker 402 is used to pick up beads from a well 410. The well 410 may be in a plate (not shown) containing 96 wells that each contains beads and/or bead mixtures. Only one well is shown in the apparatus illustrated in FIG. 4A. In a second step 412, the beads are deposited by the magnetic bead picker 402′ into a well 414. This well 414 may reside in another 96-well plate containing samples. The well 414 has a filter 416 at the bottom. The plate (not shown) containing samples and beads in well 414 is then moved to the bead incubator or washer for the next step 418 where the beads and sample in well 414′ with filter 416′ are incubated and washed.

In some embodiments, the bead washer/incubator comprises an upper chamber whereby liquid can flow in or out of the well through a filter and air can also flow in or out through the filter. The bead washer/incubator also includes a bottom chamber with a volume at least equal to the volume of the well. The bottom chamber allows both liquid and air to flow in or out of the chamber. The upper chamber also includes a liquid metering pump and a valve to direct flow either to the well or to a waste area. The upper chamber also includes an air pump and a valve to direct flow either to the well or to the vent. The lower chamber comprises similar pumps and valves as does the upper chamber. Some of these features are described in more detail in the following paragraph.

After incubation and bead washing, the well 414′ with filter 416′ is removed from the washer/incubator with the beads captured in a small volume 420 adjacent to the bottom filter 416′. The magnetic bead picker 402″ then picks up the beads from the small volume 420′ in the well 414″ with filter 416″ in step 422. In a next step 424, the magnetic bead picker 402′″ deposits the beads on a MALDI plate 404.

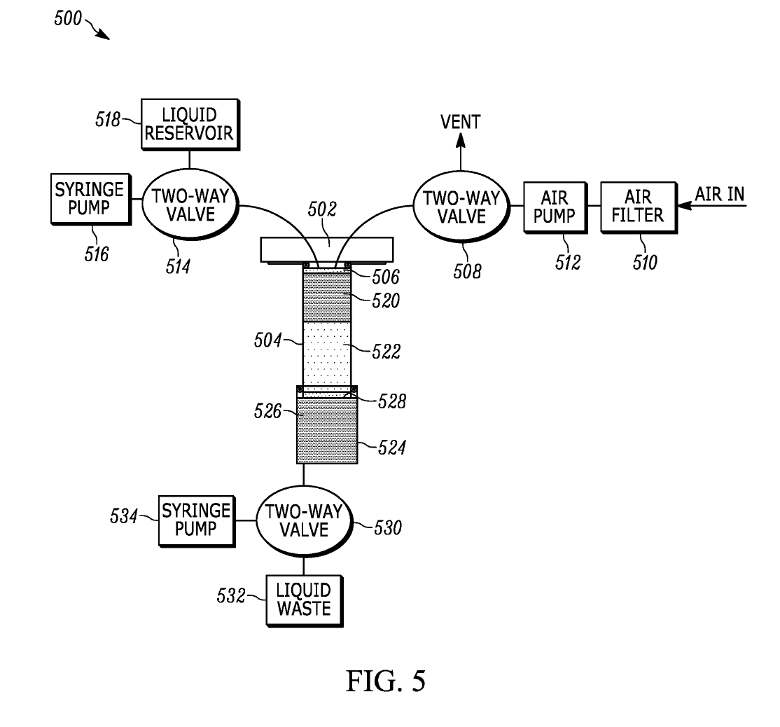

FIG. 5 illustrates a schematic diagram of an embodiment of a bead washer/incubator 500 of the present teaching. There is an upper manifold 502 attached to a well 504 with a filter 506 at the top of the well 502. This filter 506 passes air and liquid. The upper manifold 502 connects to a two-way valve 508 that connects to a vent and air supply. The input air passes through an air filter 510 and an air pump 512. The upper manifold 502 also connects to a two-way valve 514 that sends liquids to a syringe pump 516 from a liquid reservoir 518 to refill the syringe pump 516 or to direct flow from the syringe pump 516 to upper manifold 502. The well 504 contains a buffer 520 and sample plus beads 522. The lower manifold 524 forms a chamber that contains a buffer 526. There is a filter 528 at the bottom of well 504 where the well 504 connects to the lower manifold 524. The bottom of the lower manifold 524 is connected to a two-way valve 530 that goes to liquid waste 532 and a syringe pump 534.

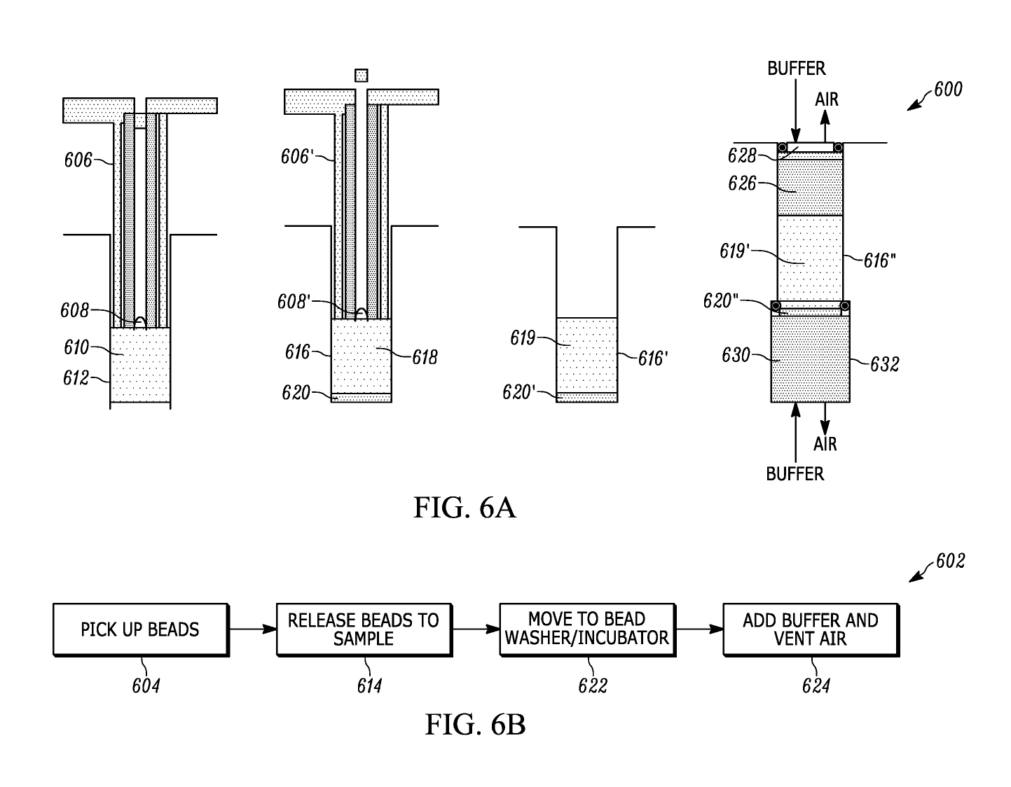

FIG. 6A illustrates an embodiment of part of a plate preparation apparatus 600 of the present teaching at different points during processing from bead pick-up through preparation for washing and incubation. FIG. 6B illustrates the steps of an embodiment of the method 602 that is associated with the apparatus of FIG. 6A. Referring to both FIGS. 6A-B, in a first step 604, the magnetic bead picker 606 picks-up a selected number or volume of beads from a mixture 610 that includes a large number of beads with different antibodies attached and then deposits them into a bead capture volume 608. In some embodiments, the large number of beads may be contained in a first well 612.

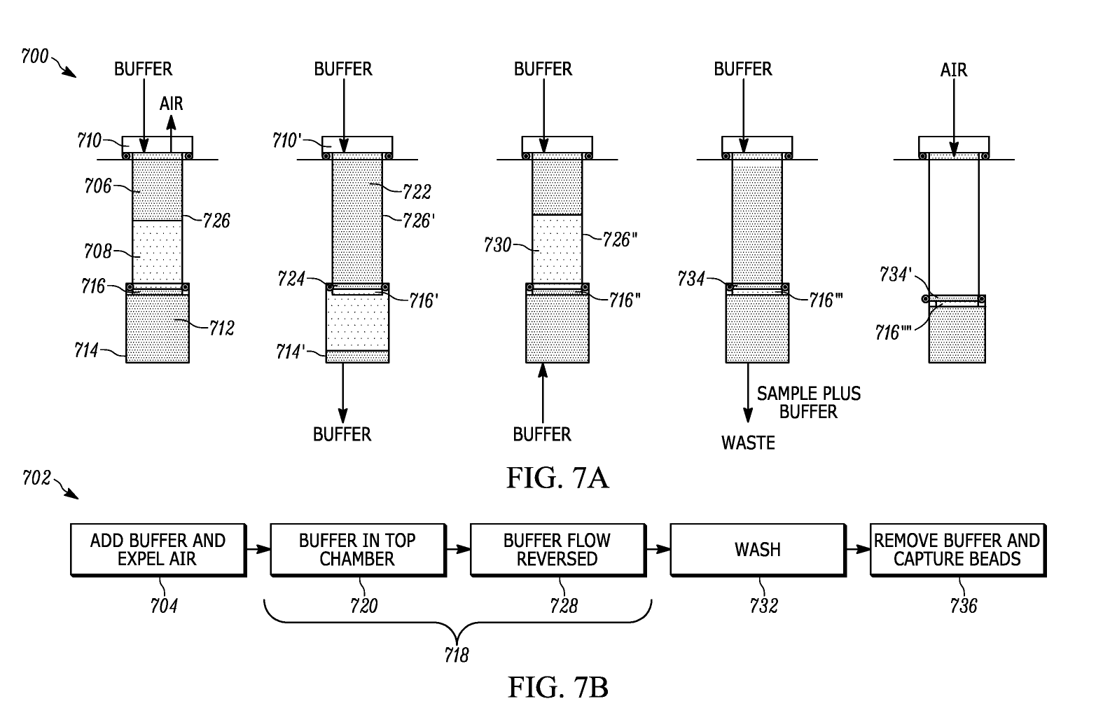

In a second step 614, the bead picker 606′ then moves to the sample well 616 and releases the beads from the bead capture volume 608′ into the sample 618. The sample well 616 includes a filter 620 at the bottom. The sample well 616 may be located on a sample plate (not shown). In a third step, the plate containing the sample well 616′ with sample and beads 619 and filter 620′ is moved to a bead washer/incubator. In a fourth step 624, a buffer 626 is added to the sample well 616″ that contains the sample plus beads 619′ and the air is expelled to fill sample well 616″ with buffer 626 up to a top filter 628 in the upper chamber. Buffer 630 is also added to the lower chamber 632 and the air is expelled. In some embodiments, the process then moves onto other steps of the method, such as illustrated in FIG. 7B.

FIG. 7A illustrates an embodiment of a portion of a plate preparation apparatus 700 according to the present teaching at different points during processing for incubating and washing the mixture of beads plus sample. FIG. 7B illustrates the steps of an embodiment of a method 702 associated with the apparatus 700 of FIG. 7A. Referring to both FIGS. 7A-B, in a first step 704, buffer 706 is added to well 726 containing sample plus beads 708 through the top chamber 710. Buffer 712 is also added to the bottom chamber 714 and air is expelled. A filter 716 at the bottom of well 726 sits atop the buffer 712 in lower chamber 714. The process then moves to incubation 718. In a second step 720 associated with incubation 718, buffer 722 is added through the upper chamber 710′ to force the sample through the filter 716′ into the lower chamber 714′ with the beads 724 being retained in the well 726′ by the filter 716′ at the bottom of the well 726′. Then, the buffer flow is reversed in a next step 728 to force the sample back through the filter 716″ and to re-suspend the beads in the sample to produce beads plus sample 730 in the well 726″. These two steps 720, 728 can be repeated as many times as necessary to assure good contact between molecules in the sample plus beads 730.

Then the method proceeds to a washing step 732. To begin the wash cycle of the washing step 732, the buffer flows from the top chamber through the bottom chamber and a valve in the liquid flow at the bottom directs the flow to the waste container (not shown). Multiple wash cycles can then be initiated by forcing buffer from the bottom chamber through the filter re-suspending the beads in the buffer. The flow can be reversed to direct the buffer flow back through the filter 716′″ with the beads 734 being retained on the filter with the flow directed to the waste container. This cycle in the washing step 732 can be repeated as many times as necessary to thoroughly wash the beads. The last step 736 in the cycle is to flow air into the top chamber to push the buffer through the filter with the beads 734′ retained on the filter 716″ in a small volume of buffer.

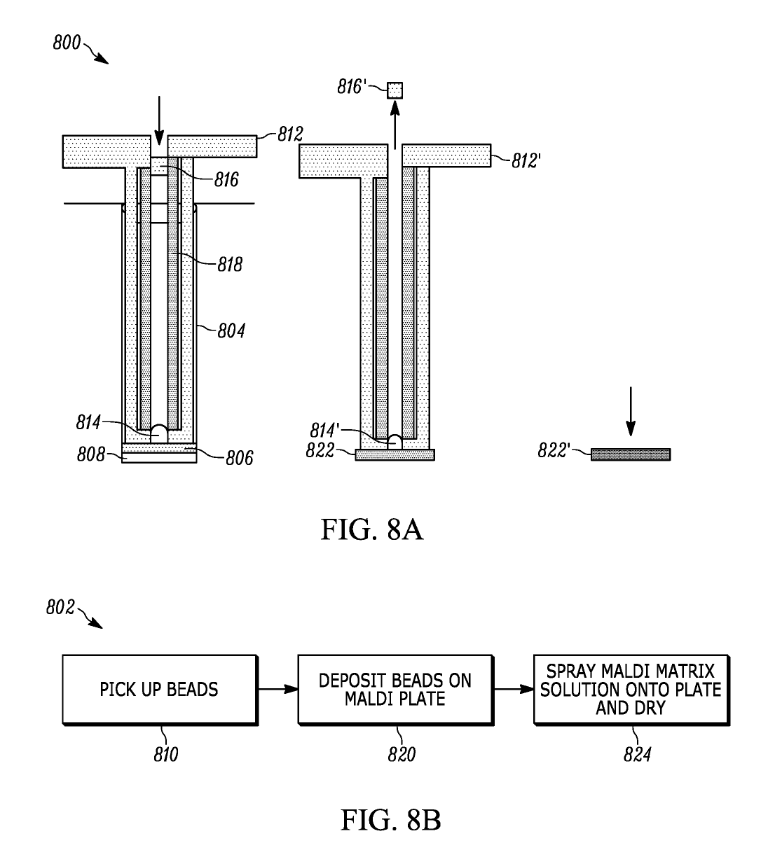

FIG. 8A illustrates an embodiment of portion of a plate preparation apparatus 800 of the present teaching at different points during processing for picking up beads through applying a MALDI matrix. FIG. 8B illustrates the steps of an embodiment of the method 802 associated with the apparatus 800 of FIG. 8A. A well 804 with the beads in a small volume 806 adjacent to the filter 808 is moved from the incubator/washer. In a first step 810, a magnetic bead picker 812 picks up the beads into a bead capture volume 814. The magnetic beads are pulled into the bead capture volume 814 by application of a magnet 816 to a soft magnetic core 818 that forms arms and concentrates the magnetic field to the bead capture volume 814. In a second step 820, the magnetic bead picker 812′ deposits the beads onto a MALDI plate 822. The magnetic beads are released from the bead capture volume 814′ by removing the magnet 816′, causing the magnetic beads to drop from the bead capture volume 814′. In a third step 824, MALDI matrix solution is applied to the plate 822′. In some embodiments, in the third step 824, a MALDI matrix solution is sprayed onto the MALDI plate 822′ and allowed to dry. In other embodiments, other known methods of applying a MALDI matrix solution to a plate containing samples are used. As the matrix solution dries, analytes non-covalently bound to capture media on the beads are released from the capture media and are incorporated into matrix crystals.

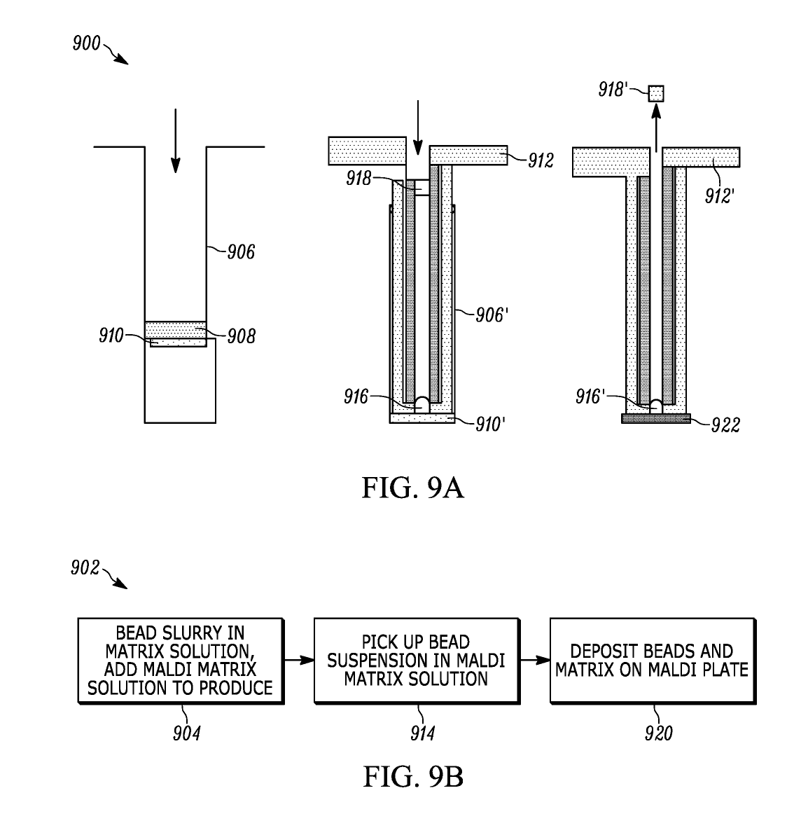

FIG. 9A illustrates another embodiment of a portion of a plate preparation apparatus 900 of the present teaching at different points during processing for picking up beads through applying a MALDI matrix. FIG. 9B illustrates the steps of an embodiment of the method 902 associated with the apparatus 900 of FIG. 9A. Referring to both FIGS. 9A and 9B, in a first step 904, a MALDI matrix solution is added to the well 906 containing the beads so that the buffer is displaced and the beads are left in a slurry 908 of MALDI matrix solution on top of the filter 910 at the bottom of the well 906. The beads saturated with matrix solution are then picked up from the well 906′ with the filter 910′ by a magnetic bead picker 912 in a second step 914. The beads saturated with MALDI matrix solution are pulled into the bead capture volume 916 by energizing the magnetic bead picker 912 by inserting the magnet 918. The beads saturated with the MALDI matrix solution are then deposited in a third step 920 on a MALDI plate 922 and allowed to dry. The beads saturated with matrix solution are released from the bead capture volume 916′ by removing the magnet 918′ from the magnetic bead picker 912′. As described above, in some embodiments an electro-magnet that is energize by application of an electric current may be used in place of the permanent magnet 918, 918′ that is inserted and removed. As the matrix solution dries, analytes non-covalently bound to capture media on the beads are released from the capture media and are incorporated into matrix crystals.

One feature of the present teaching is that it can be used to prepare samples in select individual wells of a multiple well plate. Samples can also be prepared in columns and/or rows and/or various shapes of two-dimensional arrays of a multiple well plate. In one embodiment of a method according to the present teaching, a single bead picker can be employed and the incubator/washer can accommodate one well at a time. In another embodiment, the incubator/washer accommodates one column of eight wells from a 96-well plate. Some of these embodiments do not lend themselves to automation and high throughput, but can be useful when a smaller number of samples are involved.

One feature of the present teaching is that it is possible to provide higher volume and a greater degree of automation by ganging multiple magnetic bead pickers together. In some embodiments, ninety-six magnetic bead pickers are assembled in an 8×12 array that matches a standard 96-well plate. The incubator/washer is also configured to accommodate the same 8×12 array. This arrangement can be automated and can provide relatively high throughput, but is rather inflexible in that all 96 samples are analyzed together.

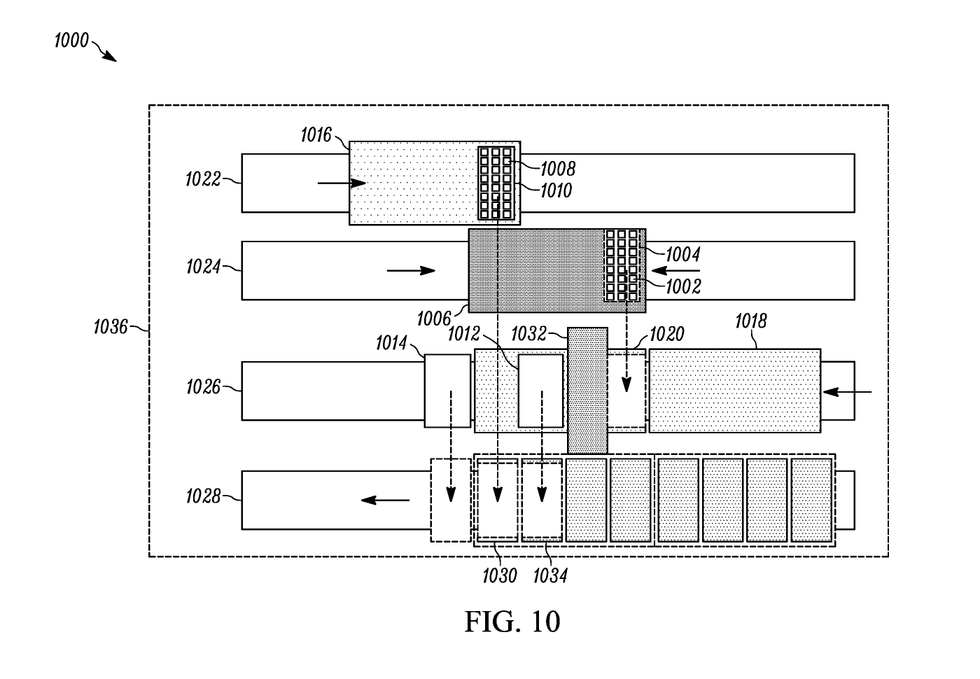

In some embodiments, the array size is smaller to provide more flexibility with measurements. FIG. 10 illustrates an embodiment of a high-volume automated MALDI plate preparation apparatus 1000 according to the present teaching. Twenty-four magnetic bead pickers 1002 are assembled in an 8×3 array 1004 that corresponds to one quarter of a 96-well plate 1006. Twenty-four pipettes 1008 are assembled in an 8×3 array 1010. In some embodiments, three 8×3 arrays 1004, 1012, 1014 of magnetic bead pickers 1002 are used. Two 8×3 arrays 1004, 1012 are used for picking and placing beads. A third, optional, 8×3 array 1014 of magnetic bead pickers 1002 is used to remove beads. Multiple 96-well plates 1006, 1016, 1018, 1020 are arranged along three parallel tracks 1022, 1024, 1026. A fourth track 1028 holds MALDI plates 1030 that are one quarter of the size of a 96 well plate. Four of these MALDI plates 1030 are ganged together along the fourth track 1028 so they are approximately equal to a size that corresponds to the 96-well plates 1018, 1020 on track three 1026. Some embodiments include one or more additional tracks (not shown) to accommodate additional plates and additional processing steps. Each track 1022, 1024, 1026, 1028 of plates supports a different processing step. In some embodiments, the first track 1022 uses the 8×3 array 1010 of pipettes 1008 to apply the MALDI matrix. The MALDI matrix is extracted from plate 1016 and the array 1010 is moved to track four 1028 to deposit the matrix. The plate 1006 on the second track 1024 supplies magnetic beads, that are picked up by the 3×8 array 1004 of pickers 1002 and subsequently deposited on the first quarter of plate 1020 on the third track 1026 that supports plates 1018, 1020 that contain samples. This is accomplished using a y-directed motion of the array 1004. A bead washer/incubator 1032 is sized to accommodate one quarter of a 96 well plate, such as plate 1020, and the washer/incubator 1032 is positioned over the third track 1026. After receiving the deposited beads, the sample plate 1020 then moves the first quarter over in the x-direction into the bead washer/incubator 1032 where the incubation and washing process occur. The first magnetic bead picker array 1004 then picks up beads from the plate 1006 containing beads and deposits them into ninety-six wells that are in the adjacent quarter of the sample plate on track three 1026, so that these beads can be moved into the washer/incubator on the next cycle. When the incubation and washer cycle on the first quarter of the sample plate 1020 is completed, the adjacent quarter of the sample plate that was just loaded with beads is introduced into the bead incubator/washer 1032 by x-directed motion along the track 1026, and the incubation washing cycle proceeds on the adjacent quarter of the sample plate. When the washed and incubated beads emerge from the washer/incubator 1032 as a result of movement of the plate 1020 in the x-direction, a second magnetic bead picker array 1012 picks up the beads from the first quarter of the sample plate 1020 and deposits them to the first MALDI plate 1034 located on the fourth track 1028. The second picker array 1012 picks and deposits beads to the fourth track 1028 simultaneously with the first magnetic bead picker array 1004 picking up beads from the plate 1006 containing beads and depositing them into the next adjacent quarter of the sample plate. In this way, the automated plate preparation apparatus 1000 efficiently and completely loads each of the plates 1020, 1018 on track three 1026 and also the plates 1030, 1034 on track four 1028 as the process progresses.

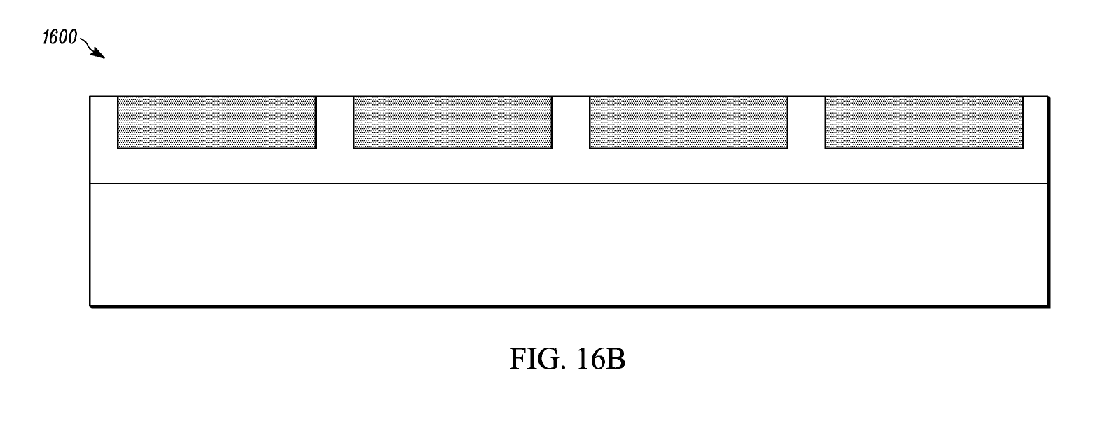

FIG. 10 also helps to illustrate how an embodiment of a complete plate preparation apparatus 1000 can carry out the various movements under computer control. A bottom deck 1036 accommodates at least four parallel tracks 1022, 1024, 1026, 1028 that allow plates to be moved as required in the x-direction. Some embodiments use five or more tracks. The fourth track 1028 includes one for the MALDI plates 1030, 1034 that are each one quarter of the 96 well plate size and are ganged in sets of four with the same dimensions as the 96-well plates used for the other tracks 1022, 1024, 1026. The configuration of MALDI plates illustrated in FIGS. 16A and 16B are described further below. Tracks are provided for sample plates 1018, 1020, the plates 1006 containing beads, and plates 1016 containing MALDI matrices. In some embodiments, plates may also be included with enzymes, such as tripsin for cleaving proteins into peptides (not shown). An upper deck (not shown explicitly in FIG. 10) includes y- and z-directed motion control for the other elements, including the magnetic bead picker arrays 1004, 1012, an 8×3 array 1010 of pipettes for transferring MALDI matrix to the MALDI sample plates. It also may include a magnetic bead remover array 1014 for cleaning the dried beads from the MALDI process. The system also includes z-directed motion for opening and closing the incubator/washer chamber 1032 and for moving the permanent magnets (not shown) from the magnetic bead picker arrays 1006, 1012, 1014.

The motion control elements required may be summarized as follows. There are up to five x-directed motion controls for moving MALDI plates 1016, sample plates 1018, 1020, bead plates 1006, matrix plates 1030, 1034 and enzyme plates (not shown). There are five y-z-directed motion controllers for the three bead picker arrays 1004, 1012, 1014, the matrix pipette array 1010, and optional enzyme pipette array (not shown). There are two z-directed motion controllers for the incubator washer 1032 that open and close the incubator washer 1032 and that move magnets (not shown). There are also three syringe pumps and three valves. This embodiment of the apparatus, therefore, requires seventeen motors, five syringe pumps, three valves, and two solenoids or motors for moving magnets in and out.

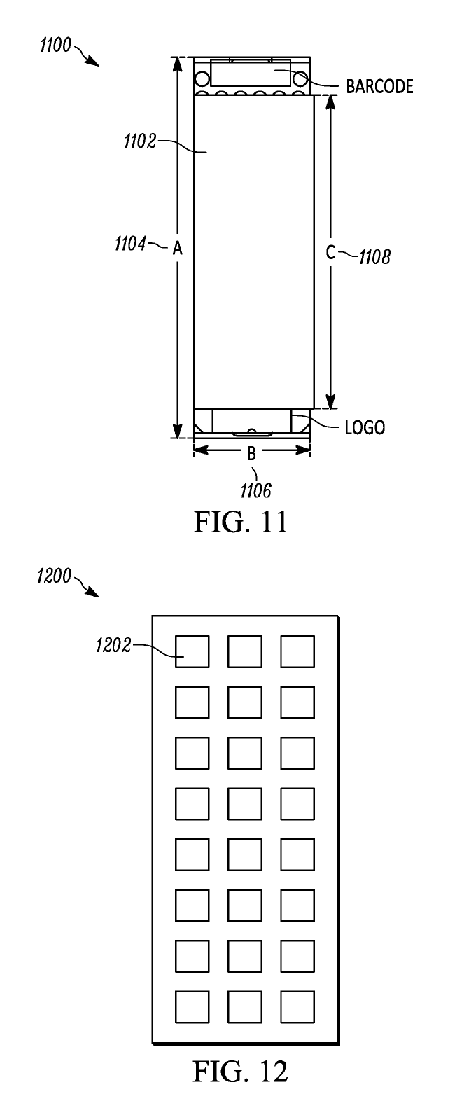

FIG. 11 illustrates an embodiment of a MALDI sample plate 1100 of the present teaching. In some embodiments, the dimensions of this plate correspond to one quarter of a 96-well plate. A region 1102 that includes an array of wells (wells not shown) suitable for use with the beads nominally 0.4 mm in diameter are shown. The plate 1100 has a width A 1104 and a length B 1106. In some embodiments, the width A is approximately 85 mm, and length B is approximately 27 mm. The region 1102 that contains wells is of length B 1106 and width C 1108. In some embodiments, B 1106 is approximately 27 mm, and C 1108 is approximately 75 mm. In some embodiments, the region 1102 comprises a gasket (details not shown) that is 0.5 mm thick with 0.5-mm-diameter holes. As such, the wells are 0.5-mm diameter and 0.5-mm deep and are spaced at intervals of 0.75 mm. This provides an array that is 36 spots wide and 96 wells long, for a total of 3456 wells. In some embodiments, the wells are formed in a silicone gasket that can be removed after the beads are dried. Note that four of the plates 1100 are equals in size to one known 384-well microtiter plate. In various methods according to the present teaching, a barcode may be used to tag information associated with the plate.

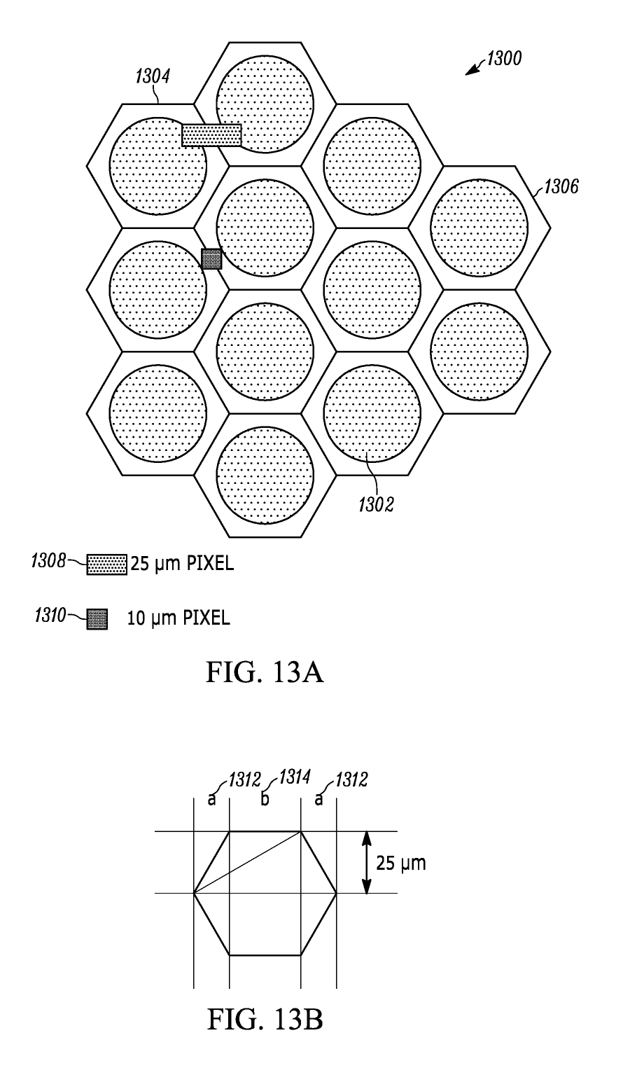

In some embodiments, the wells are grouped into spots that are formed in a second silicone gasket that can be removed after the beads are dried. FIG. 12 illustrates an embodiment of a second silicone gasket 1200 of the present teaching. This is a layout that accommodates 400 micrometer diameter beads, one bead per well. The gasket 1200 has spots 1202 arranged in a 3×8 array of spots. In some embodiments, each spot 1202 comprises a 10×10 array of wells, so the gasket 1200 includes 2400 total wells. Individual wells are not shown in FIG. 12. Some embodiments of a gasket include ninety-six spots that are approximately 3.75×3.75 mm squares on 4.5 mm centers. Each spot comprises a 5×5 array of wells, for 2400 total wells. Some embodiments of a gasket include 384 square spots of 1.5×1.5 mm dimension on 2.25 mm centers, each with a 2×2 array of wells for 1536 total wells.

While the embodiment described above in connection with the wells in the gasket of FIG. 12 is focused on employing beads that are normally 0.4 mm in diameter, the MALDI sample plate preparation apparatus is equally applicable to any size beads. FIGS. 11 and 12 support nominally one bead per well with a 400-micrometer bead. However, smaller bead sizes can be employed using large wells using multiplexing. For example, in FIG. 12 each of the 24 spots accommodates about 25,000 beads of 0.04 mm diameter and so the total number of beads is up to 600,000 beads. In various methods that use larger beads, a smaller number of beads is required for a given assay, but these methods to some extent limit the degree of multiplexing that can be done. With the smaller beads, i.e. 0.04 mm in diameter, a high degree of multiplexing is relatively convenient. For example, with the 384 spots configuration described in connection with in FIG. 12, each spot accommodates about 1000 of the small beads. This could be used, for example, to do 50-fold multiplexing, 20 beads with each bait molecule, for a total of 1000 beads in each spot.

In various other examples of multiplexing, the plurality of magnetic beads comprises at least two sets of a plurality of beads, wherein each of the at least two sets comprises a mass tag and a bait molecule that are unique to that set.

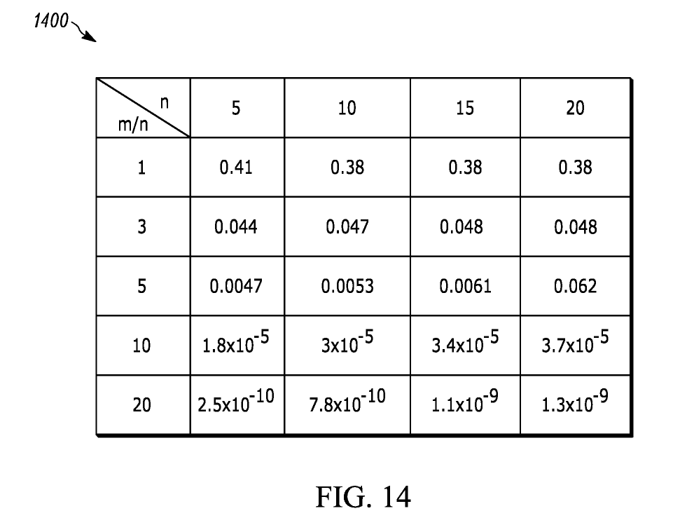

Alternatively, microwell plates that accommodate small beads in a nominally single-bead-per-well configuration can be used. For example, particular beads that are only 0.04 mm in diameter can be used with a micro well plate that has a hexagonal array of 0.04 mm in diameter wells that are up to 0.04 mm deep. FIG. 13A illustrates a diagram of a well layout 1300 for a microwell plate of the present teaching that accommodates ˜0.04-mm-diameter beads. This hexagonal well layout 1300 provides approximately 250 times as many wells as compared to the designs in each of the spots described above in connections with FIGS. 11 and 12. In the microwell embodiment shown in FIG. 13A, the microwells 1302 are 40 μm in diameter and are arranged in a regular hexagonal array 1304 with individual hexagons 1306 having a 50 μm height. FIG. 13A shows the size of a 25-μm pixel 1308 and a 10-μm pixel 1310 in relation to the size of an individual hexagon 1306 and the regular hexagon array 1304. In the example layout 1300 shown in FIG. 13A, there are nominally 462 cells/mm².

FIG. 13B shows a detailed view of one particular hexagon in the hexagonal array 1304 shown in FIG. 13A. The hexagon layout has two hexagonal dimensions, “a” 1312 and “b” 1314. In one particular example, the hexagon has a half-height of 25 μm, which corresponds to a=25 tan(30°)=14.43, and b=50 cos(30°)−a=28.86, so that b+2a=57.72 μm. The Cell Area=50² cos(30°)=2165. For a well of 40 micrometer diameter, the area=π/4 (40)²=1256 square micrometers.

In some embodiments of the ligand binding assay apparatus according to the present teaching, the laser beam in MALDI-TOF mass spectrometer is raster scanned over the surface of a microwell plate comprising the layout 1300 illustrated in FIG. 13A. In one embodiment, the raster scanning is performed at intervals of 10 μm with a 10-μm-diameter laser beam using laser repetition rate of 5 kHz, a scanning speed of 2 mm/s with 24 shots per pixel. This provides 22 total pixels per cell with 58% on the well and no significant crossover between wells. The total number of laser shots on well is 300 with a time per cell of 0.12 seconds with 462 cells per square millimeter.

One skilled in the art will appreciate that laser raster scanning schemes are also possible. For example, in one particular embodiment, the raster scanning is performed at intervals of 25 μm with a 10-μm diameter laser beam using laser repetition rate of 5 kHz, a scanning speed of 2.5 mm/s, and summing of 50 laser shots per pixel to produce 25 μm long pixels. In this particular method, the total pixels/cell ratio is about 3.5 with about half on the well and about half with significant contribution from adjacent wells. Total number of laser shots on each well is 100, with the laser irradiation time per cell equal to about 0.035 s.

In another specific method, the raster scanning is performed over the surface of microwell plate comprising the layout 1300 illustrated in FIG. 13A at intervals of 10 μm with a 10-μm diameter laser beam using a laser repetition rate of 5 kHz, scanning speed of 1 mm/s, and summing of 50 laser shots per pixel to produce 10 μm long pixels. In this particular method, the total pixels/cell ratio is about 25 with about 58% on the well having no significant contribution from adjacent wells. Total number of laser shots on the well is 600, and the laser irradiation time per cell is 0.25 seconds.

In another specific method, the raster scanning is performed over the surface of microwell plate comprising the configuration 1300 illustrated in FIG. 13A at intervals of 12.5 μm with a 10-μm diameter laser beam using laser repetition rate of 5 kHz, scanning speed of 1.25 mm/s, and summing of 50 laser shots per pixel to produce 12.5 μm long pixels. In this particular method, the total pixels/cell ratio is about 16 with about 58% on the well and with no significant contribution from adjacent wells. Total number of laser shots on each well is 400 with the laser irradiation time equal to 0.16 seconds.

If a total of m beads are sampled from a large collection where there are n beads, each with a different bait molecule attached, and the large collection is thoroughly mixed so that the probability of picking any one bead is inversely proportional to the number, n, of distinguishable beads, then the probability that any collection of m beads is missing one of the distinguishable beads is given by: F=[(n−1)/n]^{m-1}. Thus, for n=3=m, F=(2/3)²=4/9=0.44. For n=3, m=4, F=(2/3)³=8/27=0.30.

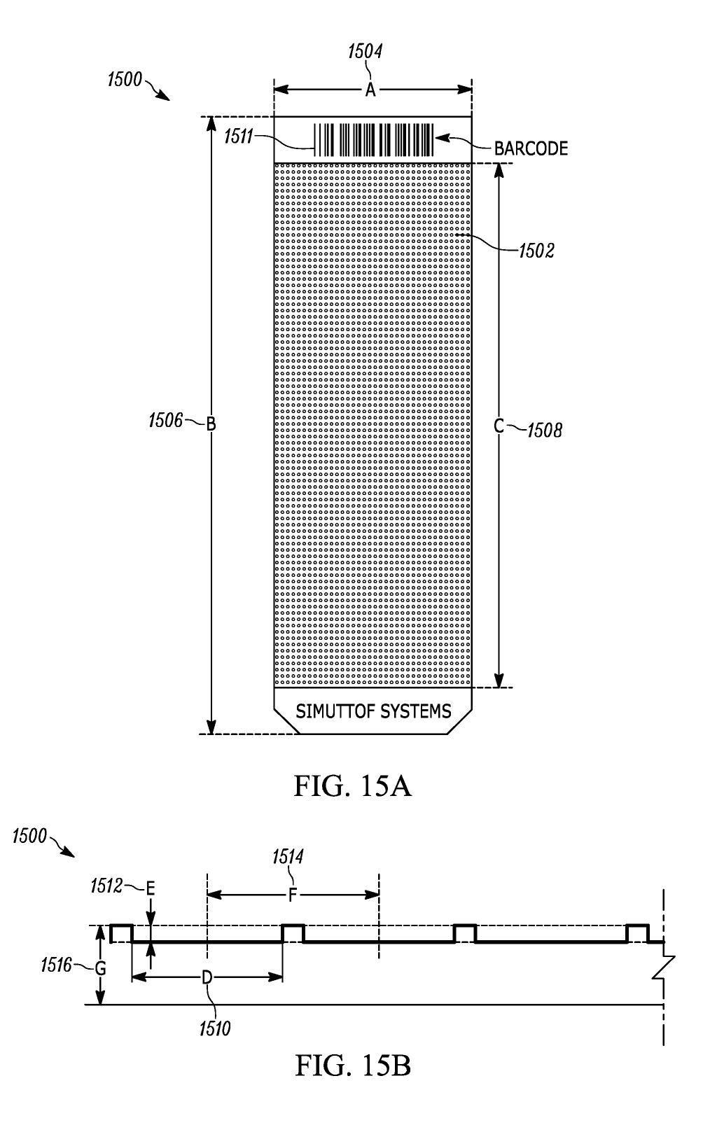

FIG. 14 illustrates a table 1400 that presents the probability of missing one of n distinguishable beads in a collection of m beads for various values of m/n and n as applies to an embodiment of the system of the present teaching. As shown by the calculation and the table 1400 in FIG. 14, the probability that one of the n beads is missed is significant unless the total number m is large compared to the number of distinguishable beads.

In the above description, it has been assumed that the mixture of beads required for a particular multiplexed assay is prepared off-line and provided in one or more wells of the bead plate. The magnetic bead picker described herein allows a predetermined number of beads to be accurately collected. For example, a single bead can be picked from a large collection of beads. This allows the mixture of beads required for a particular multiplexed assay to be mixed directly on the sample plate. For example, in some methods according to the present teaching, the first quarter of the bead plate includes of a large number of beads with a particular bait molecule attached. The next quarter includes a large number of beads with a second bait molecule. The third quarter includes a large number of beads with a third bait molecule. The fourth quarter includes a large number of beads with a fourth bait molecule. In various methods according to the present teaching, the sample plate might contain a different sample in each well. By sequential use of the bead picker, the four different beads can be introduced into each well.

In a method according to the present teaching using the MALDI sample plate described in connection with FIG. 11 with 384 spots, each with four wells in a 2×2 array allows fourfold multiplexing of 384 samples on a single small MALDI plate. The time to produce and process the data from one of these plates is less than two hours and can be as little as one hour or less. This provides the ability to analyze up to 400 samples per hour for four different analytes. Thus, the throughput is limited only by the time required to produce and prepare the samples. This means a system operating 24/7 could produce samples up to 9600 per day for four different analytes. Higher multiplexing with a smaller number of samples is also possible with very high throughput.

With the larger beads, the capacity is approximately 100 pmol per bead. A single bead may be adequate for many applications. For some applications, multiplexing is not required, for example a competing ELISA assay. For these applications, a single bead can be introduced to each sample well. After washing, the bead can be transferred to a predetermined well on the MALDI sample plate. This allows up to 3456 samples to be analyzed from a single MALDI plate. This corresponds to 36 plates each with 96 wells and provides very high throughput and low cost for these applications.

The above discussion focuses on the case in which the wells in the MALDI plate each accommodate one, and only one bead. For multiplexing, this requires that addition of MALDI matrix does not cause samples to be deposited outside of the area of the bead. This also requires the size of the laser raster pixels be less than the diameter of the well as shown in FIG. 13A. With a laser beam having a diameter of about 10 μm, the minimum pixel size is also approximately 10 μm. Thus, it is not practical to do multiplexing with beads smaller than approximately 20 μm in diameter, but much smaller diameter beads can be used with this apparatus for examples where multiplexing is not required or when a different approach to multiplexing is used. Beads as small as one micrometer in diameter can be used with this apparatus by employing a MALDI plate that has wells that can accommodate a large number of beads.

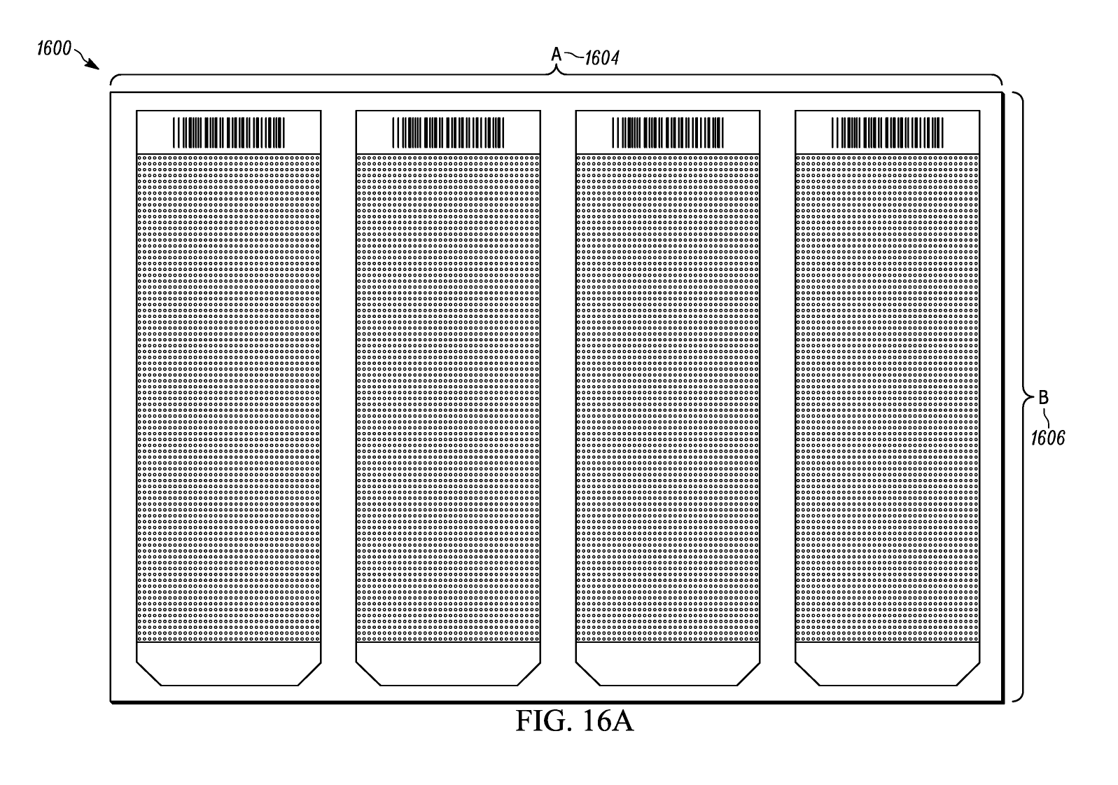

FIG. 15A illustrates a front-view of an embodiment of a MALDI sample plate 1500 that can accommodate a large number of beads of the present teaching. FIG. 15B illustrates a partial side-view of the MALDI sample plate 1500 of FIG. 15A. Referring to both FIGS. 15A and B, in some embodiments, the dimensions of this plate 1500 correspond to one quarter of a 96-well plate. A region 1502 that comprises an array of wells suitable for use with the beads nominally 0.04 mm in diameter and smaller is illustrated. The plate 1500 is of length A 1504 and width B 1506. In some embodiments, A is approximately 27 mm, and B is approximately 85.5 mm. The region 1502 that contains wells is of length A 1504 and width C 1508. In some embodiments, A 1504 is approximately 27 mm, and C 1508 is approximately 72 mm. In some embodiments, the array of wells comprise wells with diameter D 1510 of 0.6 mm, and a depth E of 0.05 mm. As such, the wells are 0.6 mm in diameter and 0.05 mm deep and are spaced at intervals of dimension F 1514 of 0.75 mm. The plate 1500 has a thickness of dimension G 1516 of 0.3 mm. This provides an array that is 36 spots wide and 96 wells long, which includes a total of 3456 wells. The well volume is 25 nL. A bead volume of a monolayer for 2.7 micrometer beads is 0.8 nL. The volume of matrix solution is approximately 50 nL/well. Using typical densities expressed in the literature, 2.7 micrometer size beads in a monolayer on a 0.6-mm-diameter spot would represent approximately 26,000 beads. A barcode 1511 may be used to identify various information. For example, the barcode 1511 can be used to identify information associated with the plate and/or associated samples and/or processing steps or other instructions.

Note that four of the plates 1500 equals approximately the same size as one known 96-well plate as shown in the embodiment illustrated in FIGS. 16A-B. FIG. 16A illustrates a top-view of an embodiment of a cassette 1600 comprising four MALDI sample plates that can accommodate a large number of beads of the present teaching wherein each of the four sample plates may be loaded into a MALDI mass spectrometer either manually or under automated or semi-automated control. FIG. 16B illustrates an end-view of a cassette 1600 comprising four MALDI sample plates of FIG. 16A. The cassette 1600 is of length A 1604 and width B 1606. In some embodiments, A is approximately 127 mm, and B is approximately 85 mm.

With the wells being 0.05 mm deep, they can accommodate any of bead diameters up to 0.05 mm in diameter. These MALDI sample plates 1500 can be formed, for example, by photo etching the array of wells in a stainless steel plate. In these cases, a monolayer of beads may be spread uniformly over the bottom of the well. MALDI matrix material may be added to each well to release biomolecules from these beads. As the matrix solution dries, the biomolecules are released from the beads incorporated into matrix crystals in the well. The MALDI plate is then transferred to the mass spectrometer and the mass spectra acquired and processed.

Referring to FIGS. 15A-B, in one embodiment, the MALDI sample plates 1500 can be formed, for example, by photo etching the array of wells in a magnetic stainless steel plate. These sample plates are held by magnets on a sled that is compatible for loading into a specified MALDI-TOF mass spectrometer. In this embodiment, a second magnetic stainless steel plate with through holes in the same pattern as MALDI plate 1500 is used. But this second plate is substantially thicker. In one example, this second plate is 0.6 mm thick and is held in place by the magnets with the holes in the second plate being aligned with the wells in sample plate 1500. This allows larger volumes of matrix solution to be added to each well. After the matrix dries, the second plate is removed before the plate 1500 is introduced into the MALDI-TOF mass spectrometer.

In some methods according the present teaching, the mass spectra acquired by laser raster scanning over a well are summed to produce an average spectrum. If each well contains beads with a number of different bait molecules attached, than the average spectrum will represent the sum of the spectra from all of the beads in the well. In developing and validating a specific bait molecule, the spectra of the captured analytes are generated. With a mixture of beads having different bait molecules, the average spectrum includes a sum of the spectra for each analyte with the relative intensities determined by the concentration of the biomolecules in the sample. Thus, spectra from each well can be deconvoluted to determine the concentration of each biomolecule. More precise quantitation can be obtained by adding a reference molecule to the sample that is captured by the bait molecule. However, this approach gives different masses than the biomolecules of interest. For example, the reference molecule may be a heavier version of the sample and the relative intensity of masses of the sample molecule compared to masses of the reference molecule can be used for accurate quantitation.

### EQUIVALENTS

While the Applicant's teaching are described in conjunction with various embodiments, it is not intended that the Applicant's teaching be limited to such embodiments. On the contrary, the Applicant's teaching encompass various alternatives, modifications, and equivalents, as will be appreciated by those of skill in the art, which may be made therein without departing from the spirit and scope of the teaching.

What is claimed is:

1. An apparatus for ligand binding assay of biological samples, the apparatus comprising:
a) a bead well configured to confine a plurality of magnetic beads, wherein each of the plurality of magnetic beads comprises an attached bait molecule;
b) a sample well comprising a filter bottom and being configured to contain samples of interest;
c) a first magnetic bead picker that captures at least some of the plurality of magnetic beads from the bead well and that releases the captured magnetic beads into the sample well;
d) an incubator that incubates the magnetic beads in the sample well, the incubation binding the bait molecules to sample molecules contained in the sample of interest;
e) a washer that washes the incubated magnetic beads, thereby removing weakly bound sample molecules while retaining magnetic beads comprising strongly bound sample molecules;
f) a sample plate that defines a plurality of wells and that is configured to load into a MALDI-TOF mass spectrometer;
g) a second magnetic bead picker that captures the magnetic beads comprising strongly bound sample molecules from the sample well and that releases the captured magnetic beads comprising strongly bound samples onto the sample plate;
h) a matrix material applicator that deposits matrix assisted laser desorption ionization (MALDI) matrix material onto a surface of the sample plate so that at least some of the strongly bound sample molecules are exposed to the MALDI matrix material;
i) a matrix assisted laser desorption ionization time-of-flight (MALDI-TOF) mass spectrometer that receives the sample plate with deposited MALDI matrix material and that performs time-of-flight mass spectrometry on the strongly bound sample molecules, thereby generating mass spectra of the sample; and
j) a computer that executes an algorithm using the mass spectra generated by the MALDI-TOF mass spectrometer to produce a ligand binding assay.

2. The apparatus for ligand binding assay of biological samples of claim 1 wherein each of the plurality of wells defined by the sample plate is dimensioned so that only one magnetic bead can be positioned in each of the plurality of wells.

3. The apparatus for ligand binding assay of biological samples of claim 1 wherein the first magnetic bead picker is configured to capture a predetermined volume of magnetic beads.

4. The apparatus for ligand binding assay of biological samples of claim 1 wherein at least one of the first magnetic bead picker and the second magnetic bead picker comprises an electro magnet.

5. The apparatus for ligand binding assay of biological samples of claim 1 wherein at least one of the first magnetic bead picker and the second magnetic bead picker comprises a permanent magnet.

6. The apparatus for ligand binding assay of biological samples of claim 1 wherein the first magnetic bead picker and the second magnetic bead picker are the same magnetic bead picker.

7. The apparatus for ligand binding assay of biological samples of claim 1 wherein the matrix material applicator comprises a sprayer that is configured to deposit MALDI matrix material onto the surface of the sample plate.

8. The apparatus for ligand binding assay of biological samples of claim 1 wherein the matrix material applicator comprises a pipette that is configured to deposit MALDI matrix material onto the surface of the sample plate.

9. The apparatus for ligand binding assay of biological samples of claim 1 wherein the MALDI-TOF mass spectrometer comprises a raster scanning ionizing laser that ionizes the strongly bound sample molecules.

10. The apparatus for ligand binding assay of biological samples of claim 1 wherein the plurality of magnetic beads comprises at least two sets of a plurality of beads, wherein each of the at least two sets comprises a mass tag and a bait molecule that are unique to that set.

11. The apparatus for ligand binding assay of biological samples of claim 1 wherein each of the plurality of beads comprises a Sepharose bead with immobilized Streptavidin.

12. The apparatus for ligand binding assay of biological samples of claim 11 wherein mass tag molecules and the bait molecules are biotinylated and are bound to Streptavidin immobilized on the Sepharose beads.

13. The apparatus for ligand binding assay of biological samples of claim 11 wherein mass tag molecules and the bait molecules covalently attach biotin to at least one of a peptide, protein or a nucleic acid.

14. The apparatus for ligand binding assay of biological samples of claim 1 wherein at least one of the plurality of beads is nominally 40 μm in diameter.

15. The apparatus for ligand binding assay of biological samples of claim 1 wherein at least one of the plurality of beads comprises biotinylated aptamers.

16. The apparatus for ligand binding assay of biological samples of claim 1 wherein at least one of the plurality of beads comprises an antibody covalently bound to the bead.

17. The apparatus for ligand binding assay of biological samples of claim 1 wherein the sample plate comprises a microwell sample plate.

18. A method for producing ligand binding assay of biological samples, the method comprising:
a) confining a plurality magnetic beads in a bead well, wherein each of the plurality of magnetic beads comprises an attached bait molecule;
b) capturing at least some of the plurality of magnetic beads from the bead well and releasing the captured magnetic beads into a sample well comprising a filter bottom configured to contain samples of interest;
c) incubating the captured magnetic beads with the sample of interest in the sample well to bind the bait molecules to sample molecules contained in the sample of interest;
d) washing the incubated magnetic beads thereby removing weakly bound sample molecules and retaining magnetic beads comprising strongly bound sample molecules;
e) capturing the washed magnetic beads comprising the strongly bound sample molecules from the sample well and releasing captured magnetic beads comprising strongly bound samples onto a sample plate that defines a plurality of wells and that is configured to load into a MALDI-TOF mass spectrometer;
f) depositing matrix assisted laser desorption ionization (MALDI) matrix material onto a surface of the sample plate so that at least some of the strongly bound sample molecules are exposed to the MALDI matrix material;
g) performing matrix assisted laser desorption ionization time-of-flight (MALDI-TOF) mass spectrometry on at least some of the strongly bound sample molecules, thereby generating mass spectra; and
h) processing the generated mass spectra to generate the ligand binding assay.

19. The method for producing ligand binding assay of biological samples of claim 18 wherein the confining the plurality magnetic beads in the bead well further comprises providing at least two sets of plurality of magnetic beads, wherein each of the at least two sets comprises a mass tag and a bait molecule that are unique to that set.

20. The method for producing ligand binding assay of biological samples of claim 18 wherein the confining the plurality magnetic beads in the bead well further comprises providing a Sepharose bead with immobilized Streptavidin.

21. The method for producing ligand binding assay of biological samples of claim 18 wherein the confining the plurality magnetic beads in the bead well further comprises biotinylating mass tag molecules and bait molecules bound to Streptavidin immobilized on the Sepharose beads.

22. The method for producing ligand binding assay of biological samples of claim 21 wherein the mass tag molecules and the bait molecules covalently attach biotin to at least one of a peptide, protein or a nucleic acid.

23. The method for producing ligand binding assay of biological samples of claim 18 wherein the confining the plurality magnetic beads in the bead well comprises providing at least some magnetic beads that are nominally approximately 40 μm in diameter.

24. The method for producing ligand binding assay of biological samples of claim 18 wherein the confining the plurality magnetic beads in the bead well comprises providing at least some magnetic beads that are nominally less than approximately 20 μm in diameter.

25. The method for producing ligand binding assay of biological samples of claim 18 wherein the providing the plurality of beads comprises providing at least some beads comprising biotinylated aptamers.

26. The method for producing ligand binding assay of biological samples of claim 18 wherein the providing the plurality of magnetic beads comprises providing at least some beads comprising an antibody covalently bound to the beads.

27. The method for producing ligand binding assay of biological samples of claim 18 wherein the washing comprises washing with a Tris pH 7.3 buffer solution.

28. The method for producing ligand binding assay of biological samples of claim 18 wherein the performing matrix assisted laser desorption ionization time-of-flight mass spectrometry comprises moving the loaded sample plate while raster scanning an ionizing laser beam.

29. The method for producing ligand binding assay of biological samples of claim 18 wherein the performing matrix assisted laser desorption ionization time-of-flight mass spectrometry comprises performing matrix assisted laser desorption ionization time-of-flight mass spectrometry in a multiplexed mode.

30. The method for producing ligand binding assay of biological samples of claim 18 wherein the processing the generated mass spectra comprises summing the generated mass spectra.

31. The method for producing ligand binding assay of biological samples of claim 18 further comprising drying the MALDI matrix material deposited on a surface of the sample plate.
